(19) United States
(12) Patent Application Publication
(10) Pub. No.: US 2015/0141404 A1
(43) Pub. Date: May 21, 2015

(54) NON-IONIC VESICLE FORMULATIONS OF CALCIUM CHANNEL BLOCKERS

(71) Applicant: CONTRACT PHARMACEUTICALS LIMITED, Mississauga (CA)

(72) Inventors: Rajiv Mathur, Mississauga (CA); Nidhi Parikh, Brampton (CA); Wennan Zhao, Mississauga (CA); Meng Zhou, Oakville (CA)

(21) Appl. No.: 14/401,766

(22) PCT Filed: May 17, 2013
(86) PCT No.: PCT/IB2013/054069

§ 371 Date: Nov. 17, 2014

Related U.S. Application Data
(60) Provisional application No. 61/648,453, filed on May 17, 2012.

(51) Int. Cl. A61K 9/00; A61K 47/10; A61K 47/28; A61K 47/36; A61K 47/44; A61K 47/32; A61K 47/34; A61K 47/26; A61K 31/554; A61K 47/12

(52) U.S. Cl. CPC A61K 9/0031; A61K 31/554; A61K 47/10; A61K 47/28; A61K 47/12; A61K 47/44; A61K 47/32; A61K 47/34; A61K 47/26; A61K 47/36
USPC 514/211.07

(57) ABSTRACT

Disclosed are non-ionic surfactant vesicle formulations of calcium channel blockers, such as diltiazem, and methods of using the formulations for treating anal disorders.

### CROSS-REFERENCE TO RELATED APPLICATIONS

The present application claims priority to U.S. Provisional Application Ser. No. 61/648,453, filed May 17, 2012, the entirety of which is incorporated herein by reference.

### FIELD

This invention generally relates to formulations of calcium channel blockers, their manufacture, and their use for the treatment of chronic anal fissures or other benign anal disorders.

### BACKGROUND

Diltiazem is a non-dihydropyridine calcium channel blocker. It is indicated orally for the treatment of chronic heart disease, particularly angina pectoris, myocardial ischemia and hypertension.

The local/topical application of diltiazem is believed to improve anal mucosal blood flow and promote healing of the fissure (Colorectal Dis. 2002 November, 4(6):430-5). By affecting vascular smooth muscle relaxation and dilatation, topical diltiazem reduces maximum resting pressure (MRP), improved healing rates and lower rates of recurrence with minimal side effects (Ann R Coll Surg Engl. 2007 July; 89(5): 472-478).

An anal fissure is a split in the skin of the distal anal canal. Young adults of both sexes are affected equally. Patients present with anal pain commonly during defecation and/or rectal bleeding. Whilst acute fissures heal spontaneously or with simple therapeutic measures, a proportion progress to form a chronic linear ulcer. Chronicity of a fissure relates to duration of greater than 6 weeks with fibres of the internal anal sphincter visible at the base of the fissure. Associated pathology may include a sentinel ‘pile’ distally and a fibro-epithelial polyp at the apex. Most anal fissures are idiopathic with no identifiable underlying disease process. There is no simple and unified theory to explain their genesis though constipation and lack of dietary fibre are implicated. Most fissures occur in the posterior midline; this may be anatomically related as there is a lack of tissue support posteriorly within the anal canal. Fissures associated with pregnancy are commonly located anteriorly and are often associated with low anal canal pressures. Other causes of fissures include Crohn's disease, syphilis, human immuno-deficiency virus (HIV) or tuberculosis. These are secondary fissures and are most appropriately treated by addressing the underlying disease process.

Most chronic anal fissures are associated with a raised internal anal sphincter (IAS) pressure and reduced vascular perfusion at the base. Current treatment has aimed at reducing resting anal pressure by diminishing sphincter tone and improving blood supply at the site of the fissure, thus promoting the healing rate. (Tech Coloproctol. 2011 June; 15(2): 135-141).

Primary anal fissures are not caused by underlying chronic disease whereas secondary anal fissures are associated with other diseases, such as chronic inflammatory intestinal diseases, human immunodeficiency virus tuberculosis, syphilis, and some neoplasms.

### SUMMARY

Among other things, the present invention recognizes that there is unmet need for new formulations of calcium channel blockers to provide sustained delivery, better dosage schemes, and/or improved patient compliance.

Among other things, the present invention recognizes the unmet need for new formulations and methods for treating anal disorders. In some embodiments, the present invention provides a non-ionic surfactant vesicle (NSV) composition of a calcium channel blocker, comprising:

about 1% to about 10% w/w of membrane forming material; and

about 1% to about 5% w/w of a calcium channel blocker.

In some embodiments, the present invention provides a method of treating an anal disorder in a subject suffering therefrom, comprising administering to the subject a therapeutically effective amount of an NSV composition, wherein the NSV composition comprises:

about 1% to about 10% w/w of membrane forming material; and

about 1% to about 5% w/w of a calcium channel blocker.

In some embodiments, in a provided method the NSV composition is applied twice a day or less. In some embodiments, in a provided method the NSV composition is applied twice a day.

### BRIEF DESCRIPTION OF THE DRAWINGS

FIG. 1(a) shows a light microscopic view of 5% diltiazem HCl NSVs under 400× magnification. FIG. 1(b) shows non-ionic surfactant vesicles (NSV) are lamellar structures that are microscopic in size. The multilayer spherical vesicles with concentric circles were observed under the light microscope in the formulations prepared using Method 1 or 2.

FIG. 2 shows comparison of release profiles for formulations with different concentrations of membrane modulator.

FIG. 3 shows comparison of release profiles for formulations with different non-ionic surfactants (membrane forming material).

### DETAILED DESCRIPTION OF CERTAIN EMBODIMENTS

Lipid vesicles, made of phospholipids or single tail amphiphiles (certain non-ionic surfactants) are membrane structures that enclose, for example, a volume of water. These non-ionic surfactant vesicles (NSVs or Niosomes or Novasomes) are self-assembly vesicles that are capable of encapsulating both hydrophobic and hydrophilic therapeutic agents and can be delivered to a subject through the skin. NSVs are analogous to, but have advantages over, liposomes as drug carriers. Some advantages include greater chemical stability, lower cost, easier storage and handling, and a reduced likelihood of becoming toxic through oxidation. Encapsulation of a drug into these Lipid vesicles can prolong the release rate of the drug on application to the skin, lower the therapeutic dose, and reduce toxicity to untargeted tissues.

In some embodiments, the present invention provides a non-ionic surfactant vesicle (NSV) composition of a calcium channel blocker, comprising:

about 1% to about 10% w/w of membrane forming material; and

about 1% to about 5% w/w of a calcium channel blocker.

In some embodiments, the membrane forming material of a provided NSV composition consists of one ingredient. In some embodiments, the membrane forming material of a provided NSV composition consists of two or more ingredients.

In some embodiments, the total amount of the membrane forming material in a provided NSV composition is about 1% to about 10% w/w. In some embodiments, the total amount of the membrane forming material in a provided NSV composition is about 1% to about 9% w/w. In some embodiments, the total amount of the membrane forming material in a provided NSV composition is about 1% to about 8% w/w. In some embodiments, the total amount of the membrane forming material in a provided NSV composition is about 1% to about 7% w/w. In some embodiments, the total amount of the membrane forming material in a provided NSV composition is about 1% to about 6% w/w. In some embodiments, the total amount of the membrane forming material in a provided NSV composition is about 1% to about 5% w/w. In some embodiments, the total amount of the membrane forming material in a provided NSV composition is about 1% to about 4% w/w. In some embodiments, the total amount of the membrane forming material in a provided NSV composition is about 1% to about 3% w/w. In some embodiments, the total amount of the membrane forming material in a provided NSV composition is about 1% to about 2% w/w. In some embodiments, the total amount of membrane forming material is about 1.5%-7% w/w. In some embodiments, the total amount of membrane forming material is about 8% w/w. In some embodiments, the total amount of membrane forming material is about 7% w/w. In some embodiments, the total amount of membrane forming material is about 6% w/w. In some embodiments, the total amount of membrane forming material is about 5% w/w. In some embodiments, the total amount of membrane forming material is about 4% w/w. In some embodiments, the total amount of membrane forming material is about 3% w/w. In some embodiments, the total amount of membrane forming material is about 3.5% w/w. In some embodiments, the total amount of membrane forming material is about 2% w/w. In some embodiments, the total amount of membrane forming material is about 1.5% w/w. In some embodiments, the total amount of membrane forming material is about 1% w/w.

Exemplary non-ionic surfactants that can be used as membrane forming material include but are not limited to fatty alcohols, fatty acids, esters of fatty alcohols, glucosides, ethoxylated fatty alcohols, sorbitan fatty acid esters, polyglycerol fatty acid esters and diesters, sucrose fatty acid esters and diesters and propylene glycol fatty acid esters and diesters. In some embodiments, an ingredient of the membrane forming material is a single-tail amphiphile. In some embodiments, an ingredient of the membrane forming material is selected from the group consisting of cetyl alcohol, ceteth-20, steareth-20, cetyl alcohol and ceteth-20 and steareth-20 (e.g., EMULCIRE, WL 2659), glyceryl monostearate (e.g., IMWITOR 491, JEECHEM GMS, HALLSTAR GMS Pure, ESTOL 1474, GELEL, LANESTA 24, CUTINA GMS, DUB GMS, CITHROL GMS, PROTACHEM GMS 450, NIKKOL MGS-A, AEC Glyceryl Stearate, GMS SE, GMS 450), polyoxyethylene 5 glyceryl stearate (e.g., TMGS-5), glyceryl distearate (e.g., HALLSTAR GDS, DUB DSG, AEC Glyceryl Distearate, NIKKOL DGS-80, ORISTAR GDS), self-emulsifying glyceryl monostearate, polyglyceryl-3-stearate (e.g., DERMOFEEL PS, CAPROL 3GS, AEC Polyglyceryl-3 Stearate), polyoxyethylene 10 stearyl ether (BRIJ S10), ceteareth 6 olivate (e.g., OLIVEM 800), polyoxyethylene 4 olivate (e.g., OLIVEM 700), and glycerol monostearate and PEG-75 stearate (e.g., GELOT 64), and combinations thereof. In some embodiments, an ingredient of the membrane forming material is selected from the group consisting of cetyl alcohol, ceteth-20, steareth-20, cetyl alcohol and ceteth-20 and steareth-20, glyceryl monostearate, polyoxyethylene 5 glyceryl stearate, glyceryl distearate, self-emulsifying glyceryl monostearate, polyglyceryl-3-stearate, polyoxyethylene 10 stearyl ether and combinations thereof. In some embodiments, an ingredient of the membrane forming material is cetyl alcohol. In some embodiments, an ingredient of the membrane forming material is ceteth-20. In some embodiments, an ingredient of the membrane forming material is steareth-20. In some embodiments, the ingredients of the membrane forming material are cetyl alcohol and ceteth-20 and steareth-20. In some embodiments, an ingredient of the membrane forming material is glyceryl monostearate 450. In some embodiments, an ingredient of the membrane forming material is polyoxyethylene 5 glyceryl stearate. In some embodiments, an ingredient of the membrane forming material is glyceryl distearate. In some embodiments, an ingredient of the membrane forming material is self-emulsifying glyceryl monostearate. In some embodiments, an ingredient of the membrane forming material is polyglyceryl-3-stearate. In some embodiments, an ingredient of the membrane forming material is polyoxyethylene 10 stearyl ether. In some embodiments, an ingredient of the membrane forming material is ceteareth 6 olivate. In some embodiments, an ingredient of the membrane forming material is polyoxyethylene 4 olivate. In some embodiments, an ingredient of the membrane forming material is glycerol monostearate. In some embodiments, an ingredient of the membrane forming material is PEG-75 stearate. In some embodiments, the ingredients of the membrane forming material are polyoxyethylene 4 olivate and glycerol monostearate and PEG-75 stearate.

In some embodiments, each ingredient of the membrane forming material of a provided NSV composition is selected from the group consisting of cetyl alcohol, ceteth-20, steareth-20, cetyl alcohol and ceteth-20 and steareth-20, glyceryl monostearate, polyoxyethylene 5 glyceryl stearate, glyceryl distearate, self-emulsifying glyceryl monostearate, polyglyceryl-3-stearate, polyoxyethylene 10 stearyl ether, ceteareth 6 olivate, polyoxyethylene 4 olivate, glycerol monostearate and PEG-75 stearate, and combinations thereof. In some embodiments, each ingredient of the membrane forming material of a provided NSV composition is selected from the group consisting of cetyl alcohol, ceteth-20, steareth-20, cetyl alcohol and ceteth-20 and steareth-20, glyceryl monostearate, polyoxyethylene 5 glyceryl stearate, glyceryl distearate, self-emulsifying glyceryl monostearate, polyglyceryl-3-stearate, polyoxyethylene 10 stearyl ether and combinations thereof. The above listed ingredients can be independently used in various amounts, both individually and in combination. Exemplary ingredients of membrane forming material and their exemplary relative amount in NSV compositions are presented in the table below:

| Ingredient | % w/w |
|---|---|
| Cetyl alcohol and ceteth-20 and steareth-20 | 1 to 5 |
| Glyceryl monostearate | 2 to 4 |
| Polyoxyethylene 5 glyceryl stearate | 1 to 3 |
| Glyceryl disteareate | 1 to 5 |
| Polyglyceryl-3-steareate | 1 to 5 |
| Polyoxyethylene 10 stearyl ether | 0.5 to 2 |
| Ceteareth 6 oliviate | 1 to 7 |
| Polyoxyethylene 4 oliviate | 1 to 7 |
| Glycerol monostearate and PEG-75 stearate | 1 to 10 |

In some embodiments, the membrane forming material of a provided NSV composition consists of two ingredients, wherein the amount of the first ingredient is about 1% to about 3% w/w, and the amount of the second ingredient is about 1% to about 5% w/w. In some embodiments, the membrane forming material of a provided NSV composition consists of two ingredients, wherein the amount of the first ingredient is about 1% to about 3% w/w, and the amount of the second ingredient is about 0.5% to about 2% w/w.

Various calcium channel blockers or the pharmaceutically acceptable salts thereof can be used in a provided composition. In some embodiments, a calcium channel blocker is diltiazem, verapamil, bepridil, nifedipine, amlodipine, felodipine, nicardipine, nisoldipine, or a pharmaceutically acceptable salt thereof, or any combination thereof. In some embodiments, a calcium channel blocker is diltiazem or a pharmaceutically acceptable salt thereof. In some embodiments, a calcium channel blocker is diltiazem HCl. In some embodiments, a, calcium channel blocker is verapamil or a pharmaceutically acceptable salt thereof. In some embodiments, a calcium channel blocker is bepridil or a pharmaceutically acceptable salt thereof. In some embodiments, a calcium channel blocker is nifedipine or a pharmaceutically acceptable salt thereof. In some embodiments, a calcium channel blocker is amlodipine or a pharmaceutically acceptable salt thereof. In some embodiments, a calcium channel blocker is felodipine or a pharmaceutically acceptable salt thereof. In some embodiments, a calcium channel blocker is nicardipine or a pharmaceutically acceptable salt thereof. In some embodiments, a calcium channel blocker is nisoldipine or a pharmaceutically acceptable salt thereof.

In some embodiments, a provided NSV composition comprises a combination of calcium channel blockers or their pharmaceutically acceptable salts thereof. In some embodiments, a provided NSV composition comprises a combination of calcium channel blockers or their pharmaceutically acceptable salts thereof, wherein the calcium channel blocker is diltiazem, verapamil, bepridil, nifedipine, amlodipine, felodipine, nicardipine, nisoldipine, or a pharmaceutically acceptable salt thereof.

In some embodiments, a provided NSV composition comprises about 1% to about 5% w/w diltiazem HCl. In some embodiments, a provided NSV composition comprises about 1% w/w diltiazem HCl. In some embodiments, a provided NSV composition comprises about 1% w/w diltiazem HCl. In some embodiments, a provided NSV composition comprises about 2% w/w diltiazem HCl. In some embodiments, a provided NSV composition comprises about 3% w/w diltiazem HCl. In some embodiments, a provided NSV composition comprises about 4% w/w diltiazem HCl. In some embodiments, a provided NSV composition comprises about 5% w/w diltiazem HCl.

In some embodiments, a provided NSV composition comprises one or more penetration enhancers. Exemplary suitable penetration enhancers are widely known in the art, including but not limited to sulphoxides (e.g., dimethylsulphoxide (DMSO)), azones (e.g., laurocapram), pyrrolidones (e.g., 2-pyrrolidone, 2P and N-methyl-2-pyrrolidone), alcohols and alkanols (e.g., ethanol or decanol, lauryl alcohol), diethylene glycol monoethyl ether, essential oils (e.g., eucalyptus, chenopodium, ylang-ylang, and L-menthol), isopropyl palmitate, isopropyl myristate, glyceryl mono laurate, oleic acid, methyl laurate, octyl salicylate, urea, sesquiterpene, glyceryl dilaurate, fatty acids (e.g., lauric acid, myristic acid and capric acid), dipropylene glycol, glyceryl monoleate, padimate O, dimethyl formamide, lecithin, glycols (e.g., propylene glycol, diethylene glycol and tetraethylene glycol). In some embodiments, a penetration enhancer is a sulphoxide. In some embodiments, a penetration enhancer is DMSO. In some embodiments, a penetration enhancer is an azone. In some embodiments, a penetration enhancer is laurocapram. In some embodiments, a penetration enhancer is a pyrrolidone. In some embodiments, a penetration enhancer is 2-pyrrolidone. In some embodiments, a penetration enhancer is 2P. In some embodiments, a penetration enhancer is N-methyl-2-pyrrolidone. In some embodiments, a penetration enhancer is an alcohol or alkanol. In some embodiments, a penetration enhancer is ethanol. In some embodiments, a penetration enhancer is a decanol. In some embodiments, a penetration enhancer is lauryl alcohol. In some embodiments, a penetration enhancer is diethylene glycol monoethyl ether. In some embodiments, a penetration enhancer is an essential oil. In some embodiments, a penetration enhancer is eucalyptus. In some embodiments, a penetration enhancer is chenopodium. In some embodiments, a penetration enhancer is ylang-ylang. In some embodiments, a penetration enhancer is L-menthol. In some embodiments, a penetration enhancer is glyceryl dilaurate or diethylene glycol monoethyl ether. In some embodiments, a penetration enhancer is a fatty acid. In some embodiments, a penetration enhancer is lauric acid. In some embodiments, a penetration enhancer is myristic acid. In some embodiments, a penetration enhancer is capric acid. In some embodiments, a penetration enhancer is dipropylene glycol. In some embodiments, a penetration enhancer is glyceryl monoleate. In some embodiments, a penetration enhancer is padimate O. In some embodiments, a penetration enhancer is dimethyl formamide. In some embodiments, a penetration enhancer is lecithin. In some embodiments, a penetration enhancer is a glycol. In some embodiments, a penetration enhancer is propylene glycol. In some embodiments, a penetration enhancer is diethylene glycol. In some embodiments, a penetration enhancer is tetraethylene glycol.

A penetration enhancer can be used either individually or in combination with one or more other penetration enhancers. In some embodiments, a provided NSV composition comprises one penetration enhancer. In some embodiments, a provided NSV composition comprises two or more penetration enhancers. In some embodiments, each penetration enhancer in a provided NSV composition is independently selected from glyceryl dilaurate and diethylene glycol monoethyl ether. In some embodiments, one penetration enhancer is used in a provided NSV composition, and the penetration enhancer is glyceryl dilaurate or diethylene glycol monoethyl ether. In some embodiments, one penetration enhancer is used in a provided NSV composition, and the penetration enhancer is glyceryl dilaurate. In some embodiments, one penetration enhancer is used in a provided NSV composition, and the penetration enhancer is diethylene glycol monoethyl ether. In some embodiments, two penetration enhancers are used in a provided NSV composition, and the penetration enhancers are glyceryl dilaurate and diethylene glycol monoethyl ether. In some embodiments, the one or more penetration enhancers in a provided composition are selected from the group consisting of sulphoxides, azones, pyrrolidones, alcohols and alkanols, diethylene glycol monoethyl ether, essential oils, isopropyl palmitate, isopropyl myristate, glyceryl monolaurate, oleic acid, methyl laurate, octyl salicylate, urea, sesquiterpene, glyceryl dilaurate, fatty acids, dipropylene glycol, glyceryl monoleate, padimate O, dimethyl formamide, lecithin, glycols and combinations thereof.

As a person having ordinary skill in the art understands, penetration enhancers can be used at various amounts in a composition. In some embodiments, the amount of glyceryl dilaurate in a provided NSV composition is about 1% to about 7%, or about 3% to about 7% w/w. In some embodiments, the amount of diethylene glycol monoethyl ether in a provided NSV composition is about 1% to about 5% w/w. In some embodiments, only one penetration enhancer is used in a provided NSV composition. In some embodiments, two or more penetration enhancers, for example, glyceryl dilaurate and diethylene glycol monoethyl ether, are used in a provided NSV composition. In some embodiments, the total amount of penetration enhancers in a provided NSV composition is about 1% to about 10%, or about 1% to about 7%, or about 3% to about 7% w/w. In some embodiments, the total amount of penetration enhancers in a provided NSV composition is about 3% to about 7% w/w. In some embodiments, the total amount of penetration enhancers in a provided NSV composition is about 3% w/w. In some embodiments, the total amount of penetration enhancers in a provided NSV composition is about 3.5% w/w. In some embodiments, the total amount of penetration enhancers in a provided NSV composition is about 4% w/w. In some embodiments, the total amount of penetration enhancers in a provided NSV composition is about 5% w/w. In some embodiments, the total amount of penetration enhancers in a provided NSV composition is about 6% w/w. In some embodiments, the total amount of penetration enhancers in a provided NSV composition is about 7% w/w.

In some embodiments, a provided NSV composition comprises one or more emollient. In some embodiments, a provided NSV composition comprises a combination of emollients. Exemplary emollients are widely described in the art, including but are not limited to cylomethicone, lanolin alcohol, acetylated lanolin alcohol, caprylic/capric triglyceride, caprylic/capric/stearic triglyceride, hydrogenated palm glycerides, butylene glycol, hexylene glycol, propylene glycol, esters of fatty alcohols, isostearic acid, lactic acid and its salts, methyl gluceth-10, methyl gluceth-20, diisopropyl adipate, methyl laurate, cetearyl ethylhexanoate, diethyl sebacate, ethylhexyl hydroxystearate, isopropyl myristate, isopropyl palmitate, cetyl palmitate, octyldodecanol, mineral oils, lauryl lactate, myristyl lactate, squalene, petrolatum, polybutene, poly vinyl pyrollidone, sorbitan isostearate, squalane, tricaprylin, urea, hyaluronic acid and its salts, glycerin, diethylene glycol monoethyl ether, acacia, butyl stearate, cholesterol, cysteine HCl, paraffinum liquidum (mineral) oil, PVP, Tocophero, zea mays (corn) starch, and sodium hyaluronate. In some embodiments, an emollient in a provided NSV composition is selected from petrolatum, mineral oil, propylene glycol, glycerine and cylomethicone.

In some embodiments, the one or more emollients in a provided composition are selected from the group consisting of cylomethicone, lanolin alcohol, acetylated lanolin alcohol, caprylic/capric triglyceride, caprylic/capric/stearic triglyceride, hydrogenated palm glycerides, butylene glycol, hexylene glycol, propylene glycol, esters of fatty alcohols, isostearic acid, lactic acid and its salts, methyl gluceth-10, methyl gluceth-20, diisopropyl adipate, methyl laurate, cetearyl ethylhexanoate, diethyl sebacate, ethylhexyl hydroxystearate, isopropyl myristate, isopropyl palmitate, cetyl palmitate, octyldodecanol, mineral oils, lauryl lactate, myristyl lactate, squalene, petrolatum, polybutene, poly vinyl pyrollidone, sorbitan isostearate, squalane, tricaprylin, urea, hyaluronic acid and its salts, glycerin, diethylene glycol monoethyl ether, acacia, butyl stearate, cholesterol, cysteine HCl, paraffinum liquidum (mineral) oil, PVP, Tocophero, zea mays (corn) starch, sodium hyaluronate, and combinations thereof. In some embodiments, the one or more emollients in a provided composition are selected from the group consisting of petrolatum, mineral oil, propylene glycol, glycerine, cylomethicone, and combinations thereof.

An emollient can be used at various amounts in a provided NSV composition. For example, petrolatum can be used at about 5% to about 15% w/w, mineral oil about 5% to about 20% w/w, propylene glycol about 1% to about 8% w/w, glycerine about 2% to about 3% w/w, and cylomethicone about 1% to about 10% w/w, either individually or in combination with one or more other emollients.

The total amount of emollients in a provided composition ranges, for example, from about 1% to about 30% w/w. In some embodiments, the total amount of emollients is about 1% to about 5% w/w. In some embodiments, the total amount of emollients is about 5% to about 10% w/w. In some embodiments, the total amount of emollients is about 10% to about 15% w/w. In some embodiments, the total amount of emollients is about 15% to about 20% w/w. In some embodiments, the total amount of emollients is about 20% to about 25% w/w. In some embodiments, the total amount of emollients is about 25% to about 30% w/w. In some embodiments, the total amount of emollients is about 5% to about 30% w/w. In some embodiments, the total amount of emollients is about 10% to about 30% w/w. In some embodiments, the total amount of emollients is about 15% to about 30% w/w. In some embodiments, the total amount of emollients is about 20% to about 30% w/w. In some embodiments, the total amount of emollients is about 5% to about 25% w/w. In some embodiments, the total amount of emollients is about 10% to about 25% w/w. In some embodiments, the total amount of emollients is about 15% to about 25% w/w. In some embodiments, the total amount of emollients is about 20% to about 25% w/w.

In some embodiments, each emollient in a provided NSV composition is selected from the group consisting of petrolatum, mineral oil, propylene glycol, glycerine and cylomethicone. In some embodiments, each emollient in a provided NSV composition is selected from the group consisting of petrolatum, mineral oil, and propylene glycol. In some embodiments, the emollients in a provided NSV composition consist of petrolatum, mineral oil, and propylene glycol. In some embodiments, the emollients in a provided NSV composition are petrolatum, mineral oil, and propylene glycol. In some embodiments, the emollients in a provided NSV composition are petrolatum (5% w/w), mineral oil (14% w/w), and propylene glycol (5% w/w). In some embodiments, the emollients in a provided NSV composition are petrolatum (5% w/w), mineral oil (14% w/w), and propylene glycol (2% w/w).

In some embodiments, a provided NSV composition comprises one or more thickeners. Exemplary suitable thickeners include but are not limited to natural gums (such as alginin, guar, locust bean, xanthan and carrageenan), starches (such as arrowroot, corn starch, katakuri starch, potato starch, sago, tapioca), pectin, gelatin, alginic acid and its salts thereof, acacia, carbomers, cellulosics (such as carboxymethylcellulose, hydroxymethyl cellulose, hydroxy ethyl cellulose, hydroxypropyl cellulose, hydroxypropyl cellulose, hydroxypropyl methylcellulose), magnesium aluminium silicates, xanthan gum, polyacrylate, stearyl alcohol and agar. In some embodiments, each thickener in a provided NSV composition is selected from the group consisting of xanthan gum, polyacrylate and stearyl alcohol. In some embodiments, a thickener is xanthan gum. In some embodiments, a thickener is polyacrylate. In some embodiments, a thickener is stearyl alcohol.

Thickeners can be used at various amounts in a provided NSV composition. For examples, xanthan gum can be used at about 0.2% to about 0.8% w/w, polyacrylate about 1% to about 4% w/w, and stearyl alcohol about 1% to about 3% w/w.

In some embodiments, one thickener is used in a provided NSV composition. In some embodiments, two or more thickeners are used in a provided NSV composition. In some embodiments, the one or more thickeners in a provided composition are selected from the group consisting of natural gums, starches, pectin, gelatin, alginic acid and its salts thereof, acacia, carbomers, cellulosics, magnesium aluminium silicates, xanthan gum, polyacrylate, stearyl alcohol, agar and combinations thereof. In some embodiments, each thickener in a provided NSV composition is selected from the group consisting of xanthan gum, polyacrylate and stearyl alcohol. In some embodiments, the one or more thickeners in a provided composition are selected from the group consisting of xanthan gum, polyacrylate, stearyl alcohol and combinations thereof.

The total amount of thickeners can be varied in a provided composition. In some embodiments, the total amount of thickeners in a provided composition is about 1% to about 5% w/w. In some embodiments, the total amount of thickeners in a provided composition is about 1% to about 2% w/w. In some embodiments, the total amount of thickeners in a provided composition is about 2% to about 3% w/w. In some embodiments, the total amount of thickeners in a provided composition is about 3% to about 4% w/w. In some embodiments, the total amount of thickeners in a provided composition is about 4% to about 5% w/w. In some embodiments, the total amount of thickeners in a provided composition is about 2% to about 4% w/w.

In some embodiments, a provided NSV composition comprises a membrane modulator. In some embodiments, a membrane modulator is a sterol. In some embodiments, a membrane modulator is cholesterol. In some embodiments, a membrane modulator is a phytosterol. Membrane modulators can be used either individually or in combination. In some embodiments, only one membrane modulator is used in a provided NSV composition. In some embodiments, the only one membrane modulator is cholesterol. In some embodiments, the only one membrane modulator is a phytosterol. In some embodiments, two or more membrane modulators are used in a provided composition. In some embodiments, the one or more membrane modulators in a provided composition are selected from the group consisting of cholesterol, sterol, phytosterol and combinations thereof. In some embodiments, the one or more membrane modulators in a provided composition are selected from the group consisting of cholesterol, phytosterol and combinations thereof.

Membrane modulators can be used at various amounts in a provided NSV composition, for example, from about 0.1% to about 4% w/w of the composition. In some embodiments, the total amount of membrane modulators in a provided NSV composition is about 0.1% to about 4% w/w. In some embodiments, the total amount of membrane modulators in a provided NSV composition is about 0.1% to about 0.2% w/w. In some embodiments, the total amount of membrane modulators in a provided NSV composition is about 0.2% to about 0.5% w/w. In some embodiments, the total amount of membrane modulators in a provided NSV composition is about 0.5% to about 1% w/w. In some embodiments, the total amount of membrane modulators in a provided NSV composition is about 1% to about 2% w/w. In some embodiments, the total amount of membrane modulators in a provided NSV composition is about 2% to about 3% w/w. In some embodiments, the total amount of membrane modulators in a provided NSV composition is about 3% to about 4% w/w. In some embodiments, the total amount of membrane modulators in a provided NSV composition is about 0.1% w/w. In some embodiments, the total amount of membrane modulators in a provided NSV composition is about 0.2% w/w. In some embodiments, the total amount of membrane modulators in a provided NSV composition is about 0.5% w/w. In some embodiments, the total amount of membrane modulators in a provided NSV composition is about 1% w/w. In some embodiments, the total amount of membrane modulators in a provided NSV composition is about 1.5% w/w. In some embodiments, the total amount of membrane modulators in a provided NSV composition is about 2% w/w. In some embodiments, the total amount of membrane modulators in a provided NSV composition is about 3% w/w. In some embodiments, the total amount of membrane modulators in a provided NSV composition is about 4% w/w.

In some embodiments, a provided composition comprises membrane forming material and one or more calcium channel blockers, and optionally one or more components independently selected from a penetration enhancer, an emollient, a thickener and a membrane modulator. In some embodiments, a provided composition comprises membrane forming material, one or more calcium channel blockers, one or more membrane modulators, and optionally one or more components independently selected from a penetration enhancer, an emollient and a thickener. In some embodiments, a provided composition comprises membrane forming material, one or more calcium channel blockers, one or more components independently selected from a penetration enhancer, an emollient and a membrane modulator, and optionally a thickener. In some embodiments, the amount of membrane forming material in the composition is about 1% to about 10% w/w. In some embodiments, the amount of the calcium channel blockers in the composition is about 1% to about 5% w/w.

In some embodiments, a provided NSV composition optionally comprises microbial preservatives, antioxidants, chelating agents and a buffering agent. Pharmaceutical compositions suitable for topical administration in and/or around the anal canal may be formulated as ointments, creams, suspensions, lotions, powders, solutions, pastes, gels, sprays, foam, oils, aerosols, suppositories or enemas.

In some embodiments, a provided NSV composition further comprises a secondary surfactant. In some embodiments, a secondary surfactant is not an ingredient of the membrane forming material. In some embodiments, a secondary surfactant is used together with a primary surfactant, such as an ingredient of the membrane forming material, to form stable membranes. In some embodiments, a secondary surfactant stabilizes the formed membrane. In some embodiments, a secondary surfactant is an ethoxylated sorbitan fatty acid ester (e.g., polysorbate 20, polysorbate 60 and polysorbate 80). In some embodiments, a secondary surfactant is polysorbate 20. In some embodiments, a secondary surfactant is polysorbate 60. In some embodiments, a secondary surfactant is polysorbate 80. In some embodiments, a secondary surfactant is polyoxyethylene 10 stearyl ether. In some embodiments, a secondary surfactant is polyoxyethylene 10 stearyl ether, and the membrane forming material is glyceryl distearate. In some embodiments, the total amount of secondary surfactants in a provided NSV composition is about 1% w/w. In some embodiments, the total amount of secondary surfactants in a provided NSV composition is about 2% w/w.

In some embodiments, a provided NSV composition has the following formulation:

about 1% about 5%(w/w) Diltiazem HCl;

about 1% to about 3% (w/w) Membrane forming material;

about 1% to about 5% (w/w) Membrane forming material;

about 1% to about 20% (w/w) emollient;

about 1% to about 3% (w/w) Penetration enhancer;

about 1% to about 3%(w/w) membrane modulator;

about 2% to about 4% (w/w) Thickener;

and balance water.

In some embodiments, a provided NSV composition has the following formulation:

about 1% about 5%(w/w) Diltiazem HCl;

about 1% to about 5% (w/w) Membrane forming material;

about 0.5% to about 2% (w/w) Membrane forming material;

about 1% to about 15% (w/w) emollient;

about 1% to about 7% (w/w) Penetration enhancer;

about 1% to about 3%(w/w) membrane modulator;

about 2% to about 4% (w/w) Thickener;

and balance water.

In some embodiments, a provided NSV composition has the following formulation:

about 1% about 5%(w/w) Diltiazem HCl;

about 1% to about 3.5% (w/w) Membrane forming material;

about 10% to about 18% (w/w) emollient;

about 1% to about 3% (w/w) Penetration enhancer;

about 1% to about 2%(w/w) membrane modulator;

about 2% to about 4% (w/w) Thickener;

and balance water.

Exemplary formulations are described below.

### Formulation 1:

| | |
|---|---|
| Diltiazem HCl- 4% | Cholesterol - 3% |
| Cetyl Alcohol and ceteth-20 and | Glyceryl Dilaurate -3% |
| steareth-20 - 3% | |
| Glyceryl Stearate - 5% | Glycerin - 2% |
| Mineral Oil - 18% | Diethylene glcyol monoethyl |
| | ether - 5% |
| Stearyl Alcohol - 3% | Polyacrylate- 2.5% |
| Petrolatum - 10% | Water - QS |

### Formulation 2:

| | |
|---|---|
| Diltiazem HCl- 4% | Cholesterol - 1.5% |
| Polyoxyethylene 5 Glyceryl Stearate - 3% | Glyceryl Dilaurate -1% |
| Polysorbate 60 - 2% | Cyclomethicone - 10% |
| Mineral Oil - 18% | Propylene Glycol - 10% |
| Stearyl Alcohol - 3% | Polyacrylate- 4% |
| Petrolatum - 12% | Water - QS |
| Polyethylene glycol - 1% | |

### Formulation 3:

| | |
|---|---|
| Diltiazem HCl- 4% | Cholesterol - 3% |
| Glyceryl Distearate - 5% | Glyceryl Dilaurate -3% |
| Polyoxyethylene 10 Stearyl | Cyclomethicone - 10% |
| Ether- 1% | |
| Mineral Oil - 14% | Diethylene glcyol monoethyl |
| | ether- 8% |
| Stearyl Alcohol - 2% | Polyacrylate- 4% |
| Petrolatum - 12% | Water - QS |
| Polysorbate 80 - 1% | |

### Formulation 4:

| | |
|---|---|
| Diltiazem HCl- 4% | Glyceryl Dilaurate -3% |
| Glyceryl monostearate - 5% | Cyclomethicone - 6% |
| Mineral Oil - 14% | Diethylene glcyol monoethyl ether- 8% |
| Stearyl Alcohol - 2% | Polyacrylate- 2% |
| Petrolatum - 12% | Xanthan gum - 0.75% |
| Polysorbate 80 - 1% | Water - QS |
| Cholesterol - 1.5% | |

### Formulation 5:

| | |
|---|---|
| Diltiazem HCl- 4% | Glyceryl Dilaurate -3% |
| Polyglyceryl-3-Steareate - 5% | Cyclomethicone - 3% |
| Mineral Oil - 14% | Diethylene glcyol monoethyl ether- 5% |
| Stearyl Alcohol - 3% | Polyacrylate- 2% |
| Petrolatum - 12% | Xanthan gum - 0.75% |
| Polysorbate 80 - 1% | Glycerin - 5% |
| Cholesterol - 1.5% | Water - QS |

### Formulation 6:

| | |
|---|---|
| Diltiazem HCl- 4% | Glyceryl Dilaurate -3% |
| Glyceryl monostearate - 3% | Cyclomethicone - 3% |
| Mineral Oil - 14% | Diethylene glcyol monoethyl ether- 5% |
| Stearyl Alcohol - 4% | Polyacrylate- 3% |
| Petrolatum - 12% | Xanthan gum - 0.75% |
| Polysorbate 80 - 0.5% | Glycerin - 4% |
| Cholesterol - 1.5% | Water - QS |

In some embodiments, a provided composition comprises about 1% to about 5% w/w diltiazem or its salts thereof, membrane forming material individually or in combination in an amount of about 1% to about 5% w/w, emollients in an amount of about 1% to about 20% w/w, penetration enhancers in an amount of about 1% to about 7% w/w, membrane modulators in an amount of 1% to 3% w/w and thickeners in an amount of 2% to 4% w/w.

Vesicles in a provided NSV composition can have different sizes, and can be either unilamellar or multilamellar. Such structural diversity, in some embodiments, provides flexibility for controlling the amount of drugs loaded, and/or the release of the loaded drugs. A provided NSV composition, when compared to traditional formulations, can provide many advantages. In some embodiments, the vesicle suspension is water based. In some embodiments, NSV can accommodate drugs having different properties; for example, both hydrophilic and lipophilic, as well as amphiphilic drugs can be loaded and delivered. In some embodiments, a provided NSV composition offers controlled release. In some embodiments, a provided NSV composition offers extended release. In some embodiments, a provided composition extends the release of diltiazem HCl over a 2 to 6 hours time period. In some embodiments, a provided NSV composition increases the stability of the entrapped drugs. In some embodiments, a provided NSV composition increases the bioavailability of drugs. In some embodiments, a provided NSV composition enhances skin penetration of drugs. In some embodiments, a provided NSV composition is used for topical use. In some embodiments, a surfactant in a provided NSV composition is biodegradable, biocompatible and non-immunogenic. In some embodiments, a provided NSV composition improves the therapeutic performance of a drug by, for example, protecting it from the biological environment, targeted delivery and/or controlled release.

In some embodiments, a provided NSV composition delivers more drug in a single dose compared to previously known compositions. In some embodiments, a provided NSV composition provides controlled release of a drug. In some embodiments, a provided NSV provides local delivery of a drug. In some embodiments, a provided NSV composition provides the same or higher local concentration while decreasing the systemic concentration of a drug. In some embodiments, a provided NSV composition reduces toxicity to untargeted tissues. In some embodiments, a provided NSV composition provides lower total dosage for a treatment. In some embodiments, a provided NSV composition provides lower total dosage for treating an anal disorder. In some embodiments, a provided NSV composition enables fewer doses per day, for example, from thrice per day to once or twice per day, for treatment of an anal disorder. In some embodiments, a provided NSV composition is used twice daily for treatment of an anal disorder. In some embodiments, a provided NSV composition enables fewer doses in a treatment regimen for an anal disorder. In some embodiments, an anal disorder is anal fissure.

As used herein, the singular forms “a”, “an”, and “the” include the plural reference unless the context clearly indicates otherwise. Thus, for example, a reference to “a peptide” includes a plurality of such peptides.

It is desirable to provide a non-surgical method for the treatment of anal fissures, particularly chronic anal fissures, which is more efficacious than methods presently available and which does not suffer from the drawbacks of the presently available nonsurgical methods for the treatment of anal fissures.

Non-ionic surfactant vesicles (NSVs) are used for the topical delivery of calcium channel blockers such as diltiazem for the treatment of benign anal disorders. The vesicles comprise a primary wall forming material. The primary wall forming material is a non-ionic or a zwiterionic surfactant. The preferred wall forming materials are polyoxyethylene glyceryl fatty acid esters, fatty alcohols, glycerol fatty acid esters or diesters, glycerol dilaurate, glucosides and mixtures thereof.

NSVs having a plurality of bi-layer membranes for encapsulating an active pharmaceutical ingredient (API) are prepared. The NSVs comprise an amphiphile, a sterol and water.

The inventors encapsulated the API Diltiazem HCl using an amphiphile selected from the group consisting of Glyceryl fatty acid mono and diesters, fatty alcohols, ethoxlated fatty alcohols, Ceteareth 6 Oliviate, PEG4 Oliviate and Polyglceryl Stearates.

Sterols, such as cholesterol or phytosterol, were used as membrane modulators. The composition may optionally comprise an emollient (such as hydrocarbon, vegetable oil, esters), or a humectant (such as water miscible Propylene glycol (PG), Glycerin, or diethylene glycol mono ethyl ether).

The composition optionally comprises a penetration enhancer, microbial preservatives, antioxidants, chelating agents and/or a buffering agent.

Pharmaceutical compositions suitable for topical administration in and/or around the anal canal may be formulated as ointments, creams, suspensions, lotions, powders, solutions, pastes, gels, sprays, foam, oils, aerosols, suppositories or enemas.

Many factors affect NSV formation, such as, the type and concentration of the amphiphiles: the type of drug and its concentration; the inclusion of varying amount of membrane modulators; changing the amphiphile-water ratio and the processing temperature and pressure.

In some embodiments, the present invention provides a method of treating an anal disorder in a subject suffering therefrom comprising administering to the subject a therapeutically effective amount of an NSV composition, wherein the NSV composition comprises:

about 1% to about 10% w/w of membrane forming material; and

about 1% to about 5% w/w of a calcium channel blocker.

In some embodiments, an anal disorder is benign. In some embodiments, an anal disorder is anal fissure.

### EXEMPLIFICATION

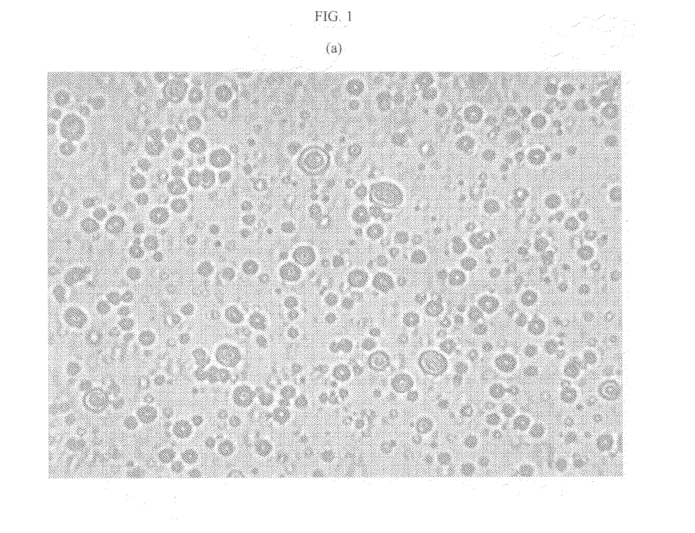

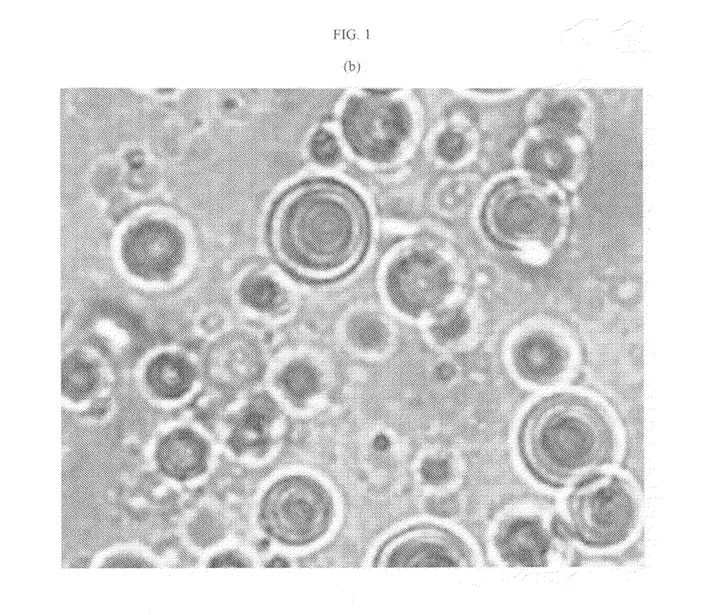

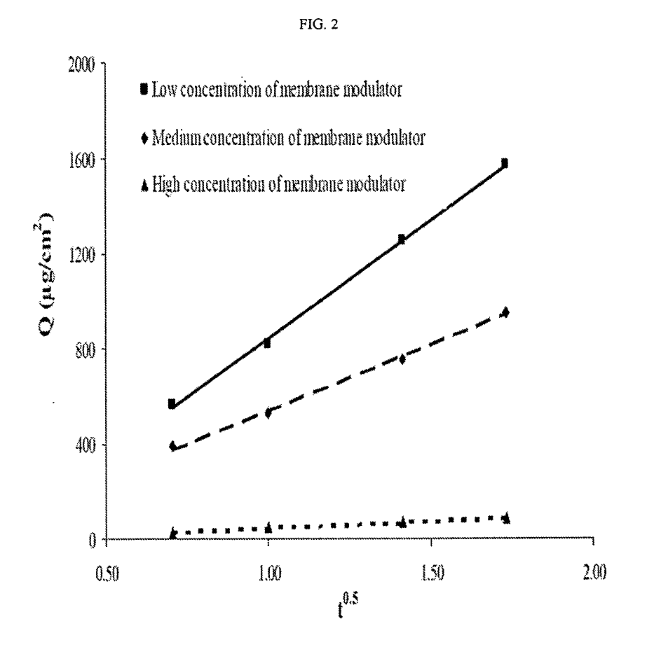

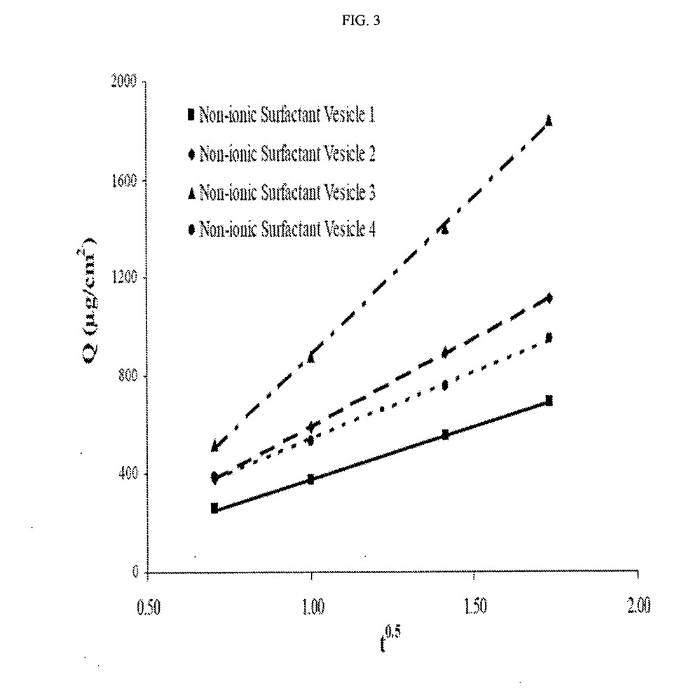

The inventors have discovered particular formulations that are optimal for producing NSVs with consistent release profiles, for use in the topical delivery of calcium channel blockers such as Diltiazem. By varying the type of amphiphile and the concentration of membrane modulator in preparation of 5% Diltiazem NSVs, a different release profile of the drug was achieved as tested by In Vitro Release Testing (TVRT) using Franz Diffusion Cells (FIGS. 2 and 3). The microscopic examination of these preparations under 400× and using polarized light showed vesicular structures with concentric circles, indicative of multilayer membrane structures (FIG. 1).

Among other things, the inventors tested the following formulae. Table 1-5 describe different formulations of the vesicles of the invention.

TABLE 1

| | |
|---|---|
| Glyceryl Distearate (HALLSTAR) | 7% |
| Glyceryl Dilaurate (JEECHEM) | 2% |
| Steareth-20 (CRODA) | 1% |
| Cholesterol (CRODA) | 1.5% |
| Mineral Oil (PENRECO) | 14% |
| Petrolatum (PENRECO) | 5% |
| Propylene Glycol (L. V LOMAS) | 5% |
| Diethylene Glycol monoethyl ether (GATEFOSSE) | 2% |
| Diltiazem HCl | 5% |
| Water | QS |

TABLE 2

| | |
|---|---|
| Glyceryl Monostearate (JEECHEM) | 7% |
| Glyceryl Dilaurate (JEECHEM) | 2% |
| Polysorbate 80 (CRODA) | 1% |
| Cholesterol (CRODA) | 1.5% |
| Mineral Oil (PENRECO) | 14% |
| Petrolatum (PENRECO) | 5% |
| Propylene Glycol (L V LOMAS) | 2% |
| Diethylene Glycol monoethyl ether (GATEFOSSE) | 5% |
| Diltiazem HCl | 1.5% |
| Water | QS |

TABLE 3

| | |
|---|---|
| Glyceryl Monostearate SE (PRIMATENE) | 3% |
| Glyceryl Dilaurate (JEECHEM) | 1.5% |
| Cholesterol (CRODA) | 1.5% |
| Mineral Oil (PENRECO) | 14% |
| Petrolatum (PENRECO) | 5% |
| Propylene Glycol (L V LOMA) | 5% |
| Diethylene Glycol monoethyl ether (GATEFOSSE) | 2% |
| Diltiazem HCl | 1.5% |
| Water | QS |

TABLE 4

| | |
|---|---|
| Polyglyceryl-3-Stearate (UNIVAR) | 7% |
| Glyceryl Dilaurate (JEECHEM) | 2% |
| Cholesterol (CRODA) | 1.5% |
| Mineral Oil (PENRECO) | 14% |
| Petrolatum (PENRECO) | 5% |
| Propylene Glycol (L.V. LOMAS) | 2% |
| Diethylene Glycol monoethyl ether (GATEFOSSE) | 5% |
| Diltiazem HCl | 5% |
| Water | QS |

TABLE 5

| | |
|---|---|
| Glyceryl Distearate (HALLSTAR) | 7% |
| Glyceryl Dilaurate (JEECHEM) | 2% |
| Polyoxyethylene 10 Stearyl Ether (CRODA) | 2% |
| Cholesterol (CRODA) | 1.5% |
| Mineral Oil (PENRECO) | 14% |
| Petrolatum (PENRECO) | 5% |
| Propylene Glycol (L V LOMAS) | 5% |
| Diethylene Glycol monoethyl ether (GATEFOSSE) | 3% |
| Diltiazem HCl | 5% |
| Water | QS |

The above formulations were then repeated by varying the concentration of cholesterol (a membrane modulator) in the range of 0.5-3.0%. The formulations were then repeated with varying amount of Diltiazem in the range of 0-10% and tested under accelerated conditions for physical stability and microscopic evaluation. All of the samples were found to be stable with vesicle size being smaller with increasing concentration of the active ingredient

The vesicles prepared form different amphiphiles and using varying concentration of cholesterol were tested for the rate of release of the active, from the product into a receptor medium, by conducting In-vitro release testing (IVRT) using Franz Diffusion cells fitted with 0.45μ Cellulose Nitrate membrane filters. The results (FIGS. 1 and 2) shows that changing the amphiphile and concentration of Cholesterol in a given vesicle formulation will result in different release profile of the encapsulated active ingredient.

More exemplary formulations were prepared and described in Formulations 1-6. In some embodiments, formulations with only 7% w/w cetereath 6 olivate, or with only 7% w/w polyoxyethylene 4 olivate, or with only 9% w/w Glycerol monostearate and PEG-75 stearate, as membrane forming material did not form non-ionic surfactant vesicles.

The following procedure is used to make vesicles of the invention. Diltiazem HCl vesicles are made using ‘shear mixing’ techniques. A lipid phase is formed by blending a primary wall forming material along with any other materials to be incorporated into the lipid bilayers, to form a homogeneous lipid phase at 70-80° C. The prepared lipid phase is blended with an aqueous phase (e.g., water, saline with other water miscible materials which will be used to hydrate the lipids) at 70-80° C., under shear mixing conditions to form the vesicles. “Shear mixing conditions”, as used herein, means a shear equivalent to a relative flow of about 50 m/second through a 1 mm orifice with a resultant pressure of about 200 psi. Such lipid vesicles can be made by a variety of devices that provides high shear for shear mixing. In the laboratory, the lipid components of each vesicle preparation are weighed out into a clean glass beaker and heated to 80° C. The materials are mixed together until a clear solution is obtained. The each clear solution is then cooled to 75-78° C. The aqueous component of each vesicle preparations is weighed into a separate clean glass beaker. Each solution is heated to 65-70° C. Each of the lipid and aqueous phase, per preparation, are transferred into 60 cc plastic syringes and connected through a 3-way stop cock with 1 mm orifice. The 2 phases are then mixed together by forcing lipid phase into aqueous phase. The resultant mixture is then transferred back and forth through stop cock at least 20 times. The syringes are then cooled to 40° C. under running cold tap water. After 5-6 more passes the product is discharged into clean dry beaker and allowed to cool to room temperature. The required thickeners are then added and mixed into the product.

In some embodiments, a provided composition is prepared by a method described below.

Method 1: Each of the lipid and aqueous phase per preparation are transferred into a 60 cc plastic syringe and connected through a 3-way stop cock with 1 mm orifice. The two phases are then mixed together by forcing lipid phase into aqueous phase. The resultant mixture is then transferred back and forth through stop cock for least 20 times. The syringes are then cooled to 40° C. under running tap water. After 5-6 more passes the product is discharged into clean dry beaker and allowed to cool to room temperature. The thickeners are then added and mixed into the product.

Method 2: The second technique applied is called ‘shear mixing’ technique. A lipid phase is formed by blending a primary wall forming material along with any other materials to be incorporated into the lipid bilayers, to form a homogeneous lipid phase at 70-80° C. The prepared lipid phase is blended with an aqueous phase (e.g., water, or other water miscible materials which will be used to hydrate the lipids) at 70-80° C., under shear mixing conditions to form the vesicles. “Shear mixing conditions”, as used herein, means a shear equivalent to a relative flow of about 50 m/second through a 1 mm orifice with a resultant pressure of about 200 psi. Such lipid vesicles can be made by a variety of devices that provides high shear for shear mixing.

IVRT Testing

The in vitro release test conditions applied for FIGS. 4-5 are shown in Table 6.

| Items | Condition |
|---|---|
| Dispersion cell | Franz cell, effective dispersive area 1.767 cm²; |
| | Diameter of 1.5 cm, internal volume of 12 mL |
| Permeation | Cellulose nitrate (0.45 μm) |
| membrane | |
| Sample quantity | 300 mg |
| Test temperature | 32° C. |
| Sampling times | 30 min, 1, 2, 3 hrs |
| Sampling quantity | 2 mL (after the sampling, 2 mL release solution |
| | heated to 32° C. will be added and the bubbles will be |
| | removed) |
| Release solution | PBS buffer solution (pH = 7.4) |

Test results are presented in FIGS. 2 and 3. FIG. 2 shows that in some embodiments, the content of membrane modulators in the formulations affects the release rate: the higher the content, the slower the release. In FIG. 2, the membrane modulator is cholesterol, the high concentration is 2.5%, the medium concentration is 1.5%, and the low concentration is 1%. The other components of the formulations are described in Table 1. FIG. 3 shows that in some embodiments, release rates can be varied by varying the type and concentration of different non-ionic surfactants. In FIG. 3, the non-ionic surfactant 1 is Cetyl Alcohol and Ceteth-20 and Steareth-20, and the corresponding formulation is Formulation 1 described above; the non-ionic surfactant 2 is Polyoxyethylene 5 Glyceryl stearate, and the corresponding formulation is Formulation 2 described above; the non-ionic surfactant 3 is Glyceryl distearate, and the corresponding formulation is Formulation 3 described above; the non-ionic surfactant vesicle 4 is Glyceryl monostearate, and corresponding the formulation is Formulation 4 described above.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

1. A non-ionic surfactant vesicle (NSV) composition of a calcium channel blocker, comprising:
about 1% to about 10% w/w of membrane forming material; and
about 1% to about 5% w/w of a calcium channel blocker.

2. The composition of claim 1, wherein the membrane forming material is selected from the group consisting of cetyl alcohol, ceteth-20, steareth-20, cetyl alcohol and ceteth-20 and steareth-20, glyceryl monostearate, polyoxyethylene 5 glyceryl stearate, glyceryl distearate, self-emulsifying glyceryl monostearate, polyglyceryl-3-stearate, polyoxyethylene 10 stearyl ether and combinations thereof.

3. The composition of claim 1 or 2, wherein the calcium channel blocker is diltiazem, verapamil, bepridil, nifedipine, amlodipine, felodipine, nicardipine, nisoldipine, or a pharmaceutically acceptable salt thereof.

4. The composition of any one of the preceding claims, wherein the calcium channel blocker is diltiazem or a pharmaceutically acceptable salt thereof.

5. The composition of any one of the preceding claims, wherein the calcium channel blocker is diltiazem hydrochloride.

6. The composition of any one of the preceding claims, further comprising one or more membrane modulators.

7. The composition of claim 6, wherein the one or more membrane modulators are selected from the group consisting of cholesterol, sterol, phytosterol and combinations thereof.

8. The composition of claim 7, wherein the membrane modulator is cholesterol.

9. The composition of any of claim 6-8, wherein the total amount of the one or more membrane modulators is about 0.1% to about 4%.

10. The composition of any one of the preceding claims, comprising about 5% diltiazem hydrochloride.

11. The composition of any one of the preceding claims, further comprising one or more penetration enhancers.

12. The composition of claim 11, wherein the one or more penetration enhancers are selected from the group consisting of sulphoxides, azones, pyrrolidones, alcohols and alkanols, diethylene glycol monoethyl ether, essential oils, isopropyl palmitate, isopropyl myristate, glyceryl monolaurate, oleic acid, methyl laurate, octyl salicylate, urea, sesquiterpene, glyceryl dilaurate, fatty acids, dipropylene glycol, glyceryl monoleate, padimate O, dimethyl formamide, lecithin, glycols and combinations thereof.

13. The composition of claim 11 or 12, wherein the total amount of the one or more penetration enhancers is about 1% to about 10% w/w.

14. The composition of any one of the preceding claims, further comprising one or more emollients.

15. The composition of claim 14, wherein the one or more emollients are selected from the group consisting of petrolatum, mineral oil, propylene glycol, glycerine, cylomethicone and combinations thereof.

16. The composition of claim 15, wherein the one or more emollients are selected from the group consisting of petrolatum, mineral oil, propylene glycol and combinations thereof.

17. The composition of any one of claims 14-16, wherein the total amount of the one or more emollients is about 1% to about 30% w/w.

18. The composition of any one of the preceding claims, further comprising one or more thickeners.

19. The composition of claim 18, wherein the one or more thickeners are selected from the group consisting of xanthan gum, polyacrylate, stearyl alcohol and combinations thereof.

20. The composition of claim 18 or 19, wherein the total amount of the one or more thickeners is about 1% to about 5% w/w.

21. The composition of any one of the preceding claims, further comprising one or more components independently selected from microbial preservatives, antioxidants, chelating agents and buffering agents.

22. The composition of any one of the preceding claims, further comprising one or more secondary surfactants.

23. The composition of claim 22, wherein the one or more secondary surfactants are selected from the group consisting of polysorbate 20, polysorbate 60, polysorbate 80, polyoxyethylene 10 stearyl ether, and combinations thereof.

24. The composition of any one of the preceding claims, wherein the composition is suitable for topical administration.

25. The composition of any one of the preceding claims, wherein the composition is formulated as an ointment, cream, suspension, lotion, powder, solution, paste, gel, spray, foam, oil, aerosol, suppository or enema.

26. A method of treating an anal disorder in a subject suffering therefrom comprising administering to the subject a therapeutically effective amount of an NSV composition, wherein the NSV composition comprises:
about 1% to about 10% w/w of membrane forming material; and
about 1% to about 5% w/w of a calcium channel blocker.

27. The method of claim 26, wherein the NSV composition is a composition of any one of claims 1-25.

28. The method of claim 26 or 27, wherein the anal disorder is anal fissure.

29. The method of any one of claims 26-28, wherein the NSV composition is applied twice per day.
